(19) United States
(12) Patent Application Publication
(10) Pub. No.: US 2026/0009058 A1
(43) Pub. Date: Jan. 8, 2026

(54) ENZYMATIC PROCESS FOR INCREASING THE SOS TRIGLYCERIDE CONTENT OF A VEGETABLE OIL

(71) Applicant: AAK AB (PUBL), Malmö (SE)

(72) Inventors: Ariana CAUSEVIC, Malmö (SE); Johanna ROSENQVIST, Malmö (SE); Kim OLOFSSON, Malmö (SE)

(21) Appl. No.: 18/993,784

(22) PCT Filed: Aug. 2, 2023
(86) PCT No.: PCT/SE2023/050770

§ 371 Date: Jan. 13, 2025

(30) Foreign Application Priority Data
Aug. 3, 2022 (SE) 2230253-3

(51) Int. Cl. C12P 7/6454; A23D 9/04; A23G 1/38; C12N 9/20

(52) U.S. Cl. CPC C12P 7/6454; A23D 9/04; A23G 1/38; C12N 9/20

(57) ABSTRACT

The present invention relates to a process for increasing the SOS triglyceride content of a vegetable oil, wherein S represents stearic acid (C18:0) and palmitic acid (C16:0) residues and O represents oleic acid (C18:1) residues, said process comprising: a) providing a reaction environment comprising: i) an sn-1,3-specific lipase immobilised on a support, ii) a vegetable oil, wherein the vegetable oil comprises at least 45% oleic acid fatty acid residues, based on total C6-C24 fatty acid residues, and wherein the vegetable oil has an oleic acid content in the sn-2 position of at least 75% by weight of the total sn-2 fatty acid residues of the vegetable oil, iii) an aliphatic alcohol ester of stearic acid, palmitic acid, or mixtures thereof, optionally mixed with stearic acid and/or palmitic acid, and iv) water; wherein the weight ratio of the aliphatic alcohol ester and optional stearic acid and/or palmitic acid (iii) to the vegetable oil (ii) is at least 4:1; and wherein the water activity of the reaction environment is in the range 0.1 to 0.6; b) heating the reaction environment to a temperature of 30-60° C. to perform transesterification, thus obtaining a mixture comprising a triglyceride phase, fatty acid esters, and optionally free fatty acids; and c) separating the fatty acid esters and free fatty acids from the mixture obtained in step (b) to obtain a triglyceride composition. The triglyceride phase may be used as a cocoa butter equivalent of a component thereof in chocolate or chocolate-like products.

### TECHNICAL FIELD OF THE INVENTION

The present invention relates to a process for increasing the SOS triglyceride content of a vegetable oil and to triglyceride compositions obtainable by the process. Triglyceride compositions which have a high content of SOS are useful as cocoa butter equivalents, or components thereof, used in the manufacture of chocolate and chocolate-like products.

### BACKGROUND OF THE INVENTION

Triglycerides comprise three fatty acid residues bonded to a glycerol backbone. Their structure can be described using “sn” notation, which stands for stereospecific numbering. In a Fischer projection of a natural L-glycerol derivative, the secondary hydroxyl group is shown to the left of C-2; the carbon atom above this then becomes C-1 and that below becomes C-3. The prefix “sn” is placed before the stem name of the compound.

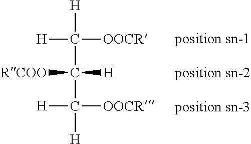

### Fischer Projection of a Natural L-glycerol Derivative.

The physical properties of a triglyceride are dependent on the nature of the fatty acid residues and their positions on the glycerol backbone. It can therefore be desirable to provide processes which alter the type and position of fatty acid residues in vegetable oils.

Triglyceride compositions which have a high SOS triglyceride content (wherein S represents stearic acid (C18:0) and palmitic acid (C16:0) residues and O represents oleic acid (C18:1) residues) are particularly commercially valuable products. SOS triglycerides are present at a high level in cocoa butter and contribute to the characteristic physical properties of chocolate. Accordingly, triglyceride compositions which have a high SOS triglyceride content can be useful as cocoa butter equivalents or components thereof.

Shea butter is used commercially as a source of SOS triglycerides, particularly where the saturated fatty acid residues S are stearic acid residues. Shea butter is obtained from the shea butter tree Butyrospermum parkii and can be fractionated to provide Shea stearin, which has a higher level of SOS triglycerides, and is therefore useful in the production of cocoa butter equivalents. The availability of Shea stearin is dependent on the supply of Shea nuts.

Enzymatic transesterification methods for producing triglyceride compositions having a high content of SOS triglycerides are known. Such methods provide a source of SOS triglycerides, which does not rely on the availability of Shea nuts.

U.S. Pat. No. 4,268,527A discloses a method for producing cocoa butter substitute by transesterification of fats and oils using sn-1,3-specific lipase and aliphatic alcohol esters of stearic acid and palmitic acid as a source of stearic and palmitic acid residues. The process utilises fats and oils which have a high oleic acid content in the sn-2 position, but most of which already contain a relatively high level of stearic and palmitic acid residues in the sn-1 and sn-3 positions, such as fractionated palm oil and sal fat. The water content of the reaction mixture is not more than 0.18% by weight. To obtain a satisfactory cocoa butter substitute, fatty acids and their aliphatic alcohol esters are distilled off and the resulting composition is subjected to fractionation.

EP2251428B1 discloses a process for making a composition having a high concentration of SOS (50-80% by weight) from triolein, for example high oleic sunflower oil, stearic acid and a sn-1,3-specific enzyme from Rhizopus oryzae. Temperatures used are preferably between 65-80° C., and the interesterified triglycerides are fractionated by solvent fractionation.

WO 2010/053244 A1 discloses cocoa butter equivalents produced by enzymatic interesterification of a wide variety of fats and oils with fatty acids or fatty acid esters using an sn-1,3-specific enzyme, distilling of reactants, and fractionation to separate the product. The molar ratio of fat or oil to fatty acid or fatty acid ester is in the range 1:2 to 1:6, the temperature used for the reaction is between 40-50° C. and water content of the oil is below 0.02%.

KR101344491B1 discloses a method for producing an oil and fat composition for cocoa butter equivalents (CBE) or cocoa butter improvers (CBI) comprising a first step of preparing a mixed oil by blending fatty acids or fatty acid esters with oils or fats; a second step of transesterification of the mixed oil by using an sn-1,3-specific enzyme at a temperature from 20° C. lower to 20° C. higher than the melting point of the mixed oil (typically 40-70° C.); and a third step of removing side-reactants through molecular distillation and fractionation after the transesterification reaction.

EP2508078A1 discloses a method for preparation of fats having a high content of POS triglycerides (in which P represents palmitic acid and S represents stearic acid) by enzymatic transesterification of vegetable fat or oil with a fatty acid or fatty acid derivative, distillation to remove the fatty acid or fatty acid derivative and then fractionation. The preferred vegetable fat or oil is palm oil or a fraction thereof.

GB2205850B discloses an enzymatic interesterification process with a low water content, which comprises subjecting a reaction liquid containing (a) a fat or oil and (b) a member selected from the group consisting of (i) another fat or oil, (ii) a fatty acid ester with a lower alcohol and (iii) a fatty acid, to the action of a lipase in the form of an immobilized enzyme catalyst, with adjusting the water concentration in the reaction liquid during the reaction.

EP 245076 A2 discloses a process for the preparation of edible fats suitable for use in confectionary, by rearrangement of unsaturated glyceride oils and fats having a high oleate content using a lipase enzyme in the presence of saturated fatty acids or esters thereof (an “acidolysis reactant”). Preferably 1 to 5 moles of acidolysis reactant per mole of oil is used, more preferably 3-5 moles per mole. In the exemplified processes, the weight ratios of acidolysis reactant to oil are 1:1 (Example 1), 1:2.5 (Example 3), and 2.4:2.5 (Example 4).

Thus, known enzymatic processes for producing triglyceride compositions which contain a high SOS triglyceride content generally involve fractionation in order to achieve an appropriate composition of triglycerides (either dry fractionation or solvent fractionation). Solvent fractionation can produce products of good quality, but the cost for building this process is high, and the use of solvents can create safety and customer acceptance issues. Dry fractionation is a more economically feasible alternative, but not as efficient in the separation of SOS triglycerides. This results in the need for product to be recirculated, which adds to process costs. More recirculation also means more formation of by-products and therefore also a decreased overall yield. More than one fractionation step may be needed to achieve the desired quality. Accordingly, both types of fractionation are complex and costly processes steps which affect the process economy.

Thus, there is a need for a more efficient enzymatic transesterification process, yielding a triglyceride composition suitable for use as a cocoa butter equivalent, or a component thereof, with improved process economy, and fewer process steps, and where there is no need for fractionation of the triglyceride composition resulting from the enzymatic transesterification. It is also desirable to provide such an enzymatic transesterification process which can utilise high oleic oil as a starting material, and which therefore does not require the use of vegetable oils which already contain a high level of stearic and/or palmitic acid, such as Shea stearin or palm oil.

### SUMMARY OF THE INVENTION

The inventors have surprisingly found that, by using particular reactants and conditions in an enzymatic transesterification process, it is possible to provide a triglyceride composition which comprises a high level of SOS triglycerides, and which can be used as a cocoa butter equivalent, or a component thereof, after simply separating fatty acid esters and free fatty acids, without the need for fractionation of the triglyceride phase.

Accordingly, the invention provides a process for increasing the SOS triglyceride content of a vegetable oil, wherein S represents stearic acid (C18:0) and palmitic acid (C16:0) residues and O represents oleic acid (C18:1) residues, said process comprising:
a) providing a reaction environment comprising:
   i) an sn-1,3-specific lipase immobilised on a support,
   ii) a vegetable oil, wherein the vegetable oil comprises at least 45% oleic acid fatty acid residues, based on total C6-C24 fatty acid residues, and wherein the vegetable oil has an oleic acid content in the sn-2 position of at least 75% by weight of the total sn-2 fatty acid residues of the vegetable oil, and
   iii) an aliphatic alcohol ester of stearic acid, palmitic acid, or mixtures thereof, optionally mixed with stearic acid and/or palmitic acid, and
   iv) water;
   wherein the weight ratio of the aliphatic alcohol ester and optional stearic acid and/or palmitic acid (iii) to the vegetable oil (ii) is at least 4:1; and
   wherein the water activity of the reaction environment is in the range 0.1 to 0.6;
b) heating the reaction environment to a temperature of 30-60° C. to perform transesterification, thus obtaining a mixture comprising a triglyceride phase, fatty acid esters, and optionally free fatty acids; and
c) separating the fatty acid esters and, where present, free fatty acids from the mixture obtained in step (b) to obtain a triglyceride composition.

The process as disclosed herein can yield a triglyceride phase which has an SOS content of at least 65% by weight of the triglyceride phase. Moreover, undesirable production of SSO triglycerides can be minimised such that the triglyceride phase of the mixture obtained in step (b) has a weight ratio of SOS triglycerides to SSO triglycerides of at least 80:1. The undesirable production of SSS triglycerides can also be minimised, such that the triglyceride phase of the mixture can have an SSS triglyceride content of ≤4% by weight of the triglyceride phase. These amounts and ratios are calculated by weight on the basis of the triglyceride phase of the mixture. The triglyceride composition is suitable for use as a cocoa butter equivalent or a component thereof.

The steps of the process above may be followed by any of the three alternative steps d1), d2) and d3), where the fatty acid esters and, optionally free fatty acids, from step c) are returned to the reaction environment of step a) with the purpose of being used as substates for transesterification:
d1) recirculating fatty acid esters and, where present, free fatty acids separated in step (c) to the reaction environment;
d2) if the aliphatic alcohol ester (iii) comprises an aliphatic alcohol ester of stearic acid, optionally mixed with stearic acid, hydrogenating fatty acid esters and, where present, free fatty acids separated in step (c) and recirculating the hydrogenated fatty acid esters and free fatty acids to the reaction environment;
d3) separating fatty acid esters and, where present, free fatty acids separated in step (c) into:
   a first fraction comprising stearate and/or palmitate ester, and optionally stearic acid and/or palmitic acid; and
   a second fraction comprising oleate ester and optionally oleic acid; and recirculating the first fraction to the reaction environment.

Where the aliphatic alcohol ester (iii) comprises an aliphatic alcohol ester of stearic acid, optionally mixed with stearic acid, the process may also comprise a further step (e3) of hydrogenating the second fraction to provide stearate ester and optionally stearic acid and recirculating the hydrogenated second fraction to the reaction environment.

The vegetable oil (ii) may be selected from High Oleic Rapeseed/canola oil, Olive oil, High Oleic Soybean oil, High Oleic Sunflower oil, High Oleic Safflower oil, Shea oil, or Rapeseed oil, and/or fractions or combinations thereof. Preferably the vegetable oil (ii) is selected from High Oleic Sunflower oil, High Oleic Safflower oil, and/or combinations or fractions thereof.

The process may further comprise using the triglyceride composition obtained in step (c) as a cocoa butter equivalent or a component thereof in the manufacture of a chocolate or chocolate-like product.

The invention also provides triglyceride composition obtainable by the process.

The invention also provides a chocolate or chocolate-like product comprising the triglyceride composition.

The invention also provides a chocolate or chocolate-like product comprising the CBE.

### Definitions

As used herein, the term “vegetable” shall be understood as originating from a plant or a single cell organism. Thus, vegetable oil or vegetable triglycerides are still to be understood as vegetable oil or vegetable triglyceride if all the fatty acids used to obtain said triglyceride or oil is of plant or single cell organism origin.

The term “oil” as used herein refers to glyceride fats and oils containing fatty acid acyl groups and does not imply any particular melting point. The term “fat” is used synonymously with “oil” herein.

As used herein, the term “oils derived therefrom” encompasses any processed oils, i.e. oils that have undergone a process. For example, this term includes any fraction of oils, i.e. oils that have undergone fractionation.

As used herein, the term “fatty acid” encompasses free fatty acids and fatty acid residues in triglycerides.

Using the nomenclature CX means that the fatty acid comprises X carbon atoms, e.g. a C16-fatty acid has 16 carbon atoms while a C18-fatty acid has 18 carbon atoms.

Using the nomenclature CX: Y means that the fatty acid comprises X carbon atoms and Y double bonds, e.g. a C16:0 fatty acid has 16 carbon atoms and 0 double bonds while a C18:1 fatty acid has 18 carbon atoms and 1 double bond.

As used herein the abbreviations “SOS” and “SSO” encompasses triglycerides (TAG's), where S denotes the saturated fatty acid esters (FAE's) palmitate (C16:0) or stearate (C18:0), and O the unsaturated oleate (C18:1). Thus “SOS” denotes monounsaturated triglycerides with one O in the sn-2 position, and one S (either a stearic acid or palmitic acid residue) in each of the sn-1 and sn-3 positions. “SSO” denotes asymmetric monounsaturated triglycerides with one O in either the sn-1 or sn-3 positions, one S in the sn-2 position, and one S in either the sn-1 or sn-3 positions.

Cocoa butter equivalents, are well-known in the art as edible fats, typically made up of one or more vegetable fats, having a composition and properties that are similar to cocoa butter, and that are compatible with cocoa butter and that have no significant effect on the behaviour of a chocolate in which they are used.

As used herein, “%” or “percentage” relates to weight percentage i.e. wt. % or wt.-% if nothing else is indicated.

As used herein the expression “water activity” is defined as the partial vapor pressure of water in the oil divided by the vapor pressure of pure water at the same temperature. At equilibrium the water activity is equal to the relative humidity. One simple and commonly used method for adjusting the water activity is pre-equilibration with saturated salt solutions through the vapor phase in sealed containers (R. H. Valivety, P. J. Halling, A. R. Macrae, Reaction rate with suspended lipase catalyst shows similar dependence on water activity in different organic solvents, Biochim. Biophys. Acta (BBA)/Protein Struct. Mol. (1992) and H. L. Goderis, G. Ampe, M. P. Feyten, B. L. Fouwe, W. M. Guffens, S. M. Van Cauwenbergh, P. P. Tobback, Lipase-catalyzed ester exchange reactions in organic media with controlled humidity, Biotechnol. Bioeng. 30 (1987) 258-266). Another method is sparging of the substrate with dry or humid air or nitrogen gas for removal or supply of water (K. Won, Sun Bok Lee, Computer-aided control of water activity for lipase-catalyzed esterification in solvent-free systems, Biotechnol. Prog. 17 (2001) 258-264 and A. E. V. Petersson, P. Adlercreutz, B. Mattiasson, A water activity control system for enzymatic reactions in organic media, Biotechnol. Bioeng. 97 (2007) 235-241).

As used herein the expression “reaction environment” is the environment where the enzymatic transesterification takes place and comprises the sn-1,3-specific lipase immobilized on a support, water, the reactants, i.e. the vegetable oil and the aliphatic alcohol ester of stearic acid, palmitic acid, or mixtures thereof, optionally mixed with stearic acid and/or palmitic acid.

As used herein the expression “feed” is defined as the ingoing sum in weight of substrates for the sn-1,3-specific lipase, i.e. the sum of weight of the vegetable oil (ii) and the aliphatic alcohol ester of stearic acid, palmitic acid, or mixtures thereof, optionally mixed with stearic acid and/or palmitic acid (iii).

As used herein, the expression “substrate ratio” is the weight ratio of the aliphatic alcohol ester of stearic acid, palmitic acid, or mixtures thereof, optionally mixed with stearic acid and/or palmitic acid (iii) to the vegetable oil (ii).

### BRIEF DESCRIPTION OF THE DRAWINGS

FIG. 1 depicts process diagrams for three different embodiments of the process of the invention.

FIG. 1A depicts a process in which fatty acid esters (FAEs) and free fatty acids (FFAs) which are separated from the triglyceride phase following the enzymatic transesterification reaction are recirculated to the reaction environment.

FIG. 1B depicts a process in which fatty acid esters (FAEs) and free fatty acids (FFAs) which are separated from the triglyceride phase following the enzymatic transesterification reaction are hydrogenated before being recirculated to the reaction environment.

FIG. 1C depicts a process in which fatty acid esters (FAEs) and free fatty acids (FFAs) which are separated from the triglyceride phase following the enzymatic transesterification reaction are separated into a first fraction comprising stearate and/or palmitate ester, and optionally stearic acid and/or palmitic acid and a second fraction comprising oleate ester and optionally oleic acid. The first fraction is recirculated to the reaction environment and the second fraction is hydrogenated before also being recirculated to the reaction environment.

### DETAILED DESCRIPTION OF THE INVENTION

When describing the below embodiments, the present invention envisages all possible combinations and permutations of the below described embodiments with the above disclosed aspects.

The invention relates to a process for increasing the SOS triglyceride content of a vegetable oil, wherein S represents stearic acid (C18:0) and palmitic acid (C16:0) residues and O represents oleic acid (C18:1) residues, said process comprising:
a) providing a reaction environment comprising:
   i) an sn-1,3-specific lipase immobilised on a support,
   ii) a vegetable oil, wherein the vegetable oil comprises at least 45% oleic acid fatty acid residues, based on total C6-C24 fatty acid residues, and wherein the vegetable oil has an oleic acid content in the sn-2 position of at least 75% by weight of the total sn-2 fatty acid residues of the vegetable oil,
   iii) an aliphatic alcohol ester of stearic acid, palmitic acid, or mixtures thereof, optionally mixed with stearic acid and/or palmitic acid, and
   iv) water;
   wherein the weight ratio of the aliphatic alcohol ester and optional stearic acid and/or palmitic acid (iii) to the vegetable oil (ii) is at least 4:1; and
   wherein the water activity of the reaction environment is in the range 0.1 to 0.6;
b) heating the reaction environment to a temperature of 30-60°° C. to perform transesterification, thus obtaining a mixture comprising a triglyceride phase, fatty acid esters, and optionally free fatty acids; and
c) separating the fatty acid esters and free fatty acids from the mixture obtained in step (b) to obtain a triglyceride composition.

The triglyceride phase of the mixture obtained in step (b) may have an SOS triglyceride content of at least 65% by weight of the triglyceride phase and a weight ratio of SOS triglycerides to SSO triglycerides of at least 80:1. The SSO content of the triglyceride phase may be ≤1.2% by weight of the triglyceride phase. The SSS content of the triglyceride phase may be ≤4% by weight of the triglyceride phase, preferably ≤3% by weight of the triglyceride phase, more preferably ≤2% by weight of the triglyceride phase and most preferably ≤1.5% by weight of the triglyceride phase. The composition can therefore be used as a cocoa butter equivalent, or a component thereof, without the need for further fractionation of the triglyceride phase.

The SOS triglyceride, SSO triglyceride and SSS triglyceride content of the triglyceride phase can be determined using a non-aqueous reversed-phase HPLC method. A suitable method is described in “Non-aqueous reversed phase liquid chromatography with charged aerosol detection for quantitative lipid analysis with improved accuracy”, Causevic, A. et al., Journal of Chromatography A, Vol. 1652, 2021, pages 1-11.

The reaction environment for the enzymatic transesterification comprises an sn-1,3-specific lipase immobilised on a support. This enzyme effects transesterification at the sn-1 and sn-3 positions of the vegetable oil, thus replacing fatty acid residues at these positions in the vegetable oil with stearic and palmitic acid residues from the aliphatic alcohol esters of stearic acid or palmitic acid and the optional free fatty acids. In one or more embodiments the sn-1,3-specific lipase (i) is a microbial lipase, for example a bacterial or fungal lipase. Preferably, the sn-1,3-specific lipase is derived from a fungal species, especially the species Rhizopus oryzae, Thermomyces lanuginosus, or Rhizomucor miehei. Lipases derived from these species have been found to be particularly suitable for the transesterification process of the invention in terms of specificity, reaction rate and robustness.

The immobilization of enzymes on supports is well known in the art, and immobilized sn-1,3-specific lipases are commercially available from various suppliers. Immobilization of sn-1,3-specific lipases on support materials has been found to improve enzyme performance, thus providing higher levels of SOS triglycerides. A variety of support materials are known, such as various polymers and silica. In an embodiment of the invention, the support material on which the sn-1,3-specific lipase is immobilized is hydrophobic. One particularly useful enzyme is immobilized Lipase DF “Amano” IM from Rhizopus oryzae (available from Amano Enzyme).

The vegetable oil (ii) used in the process of the invention should have a fatty acid composition with a relatively high level of oleic acid. The fatty acid composition of an oil or fat can be determined by a gas chromatographic analysis of the methyl ester derivatives, prepared by transesterification. The technique of gas-liquid chromatography (GLC), also referred to as gas chromatography (GC), is a form of partition chromatography in which the mobile phase is a gas and the stationary phase is a liquid. The sample is volatilised during injection and an equilibrium is formed between the gas phase and the liquid phase, which is fixed at the inner wall of the column. When the sample contains different components, they diffuse into the liquid phase to varying degrees according to their individual equilibrium constant, and so travel down the column at different rates. This results in different retention times, and thus a physical separation. The separated components emerge from the end of the column exhibiting peaks of concentration, ideally with a Gaussian distribution. These peaks are detected by the Flame Ionization Detector (FID), which converts the concentration of the component in the gas phase into an electrical signal, which is amplified and passed to a continuous recorder, so that the progress of the separation can be monitored and quantified. A suitable method is IUPAC method 2.304.

The vegetable oil (ii) which is used as a reactant for the transesterification should comprise at least 45% oleic acid (C18:1) fatty acid residues, based on total C6-C24 fatty acid residues of the vegetable oil which is provided in the reaction environment. Preferably, the vegetable oil comprises at least 50% oleic acid (C18:1) fatty acid residues, more preferably at least 60% oleic acid (C18:1) fatty acid residues, and most preferably at least 70% oleic acid (C18:1) fatty acid residues. One advantageous feature of the invention is that vegetable oils which have a high level of oleic acid not only in the sn-2 position, but also in the sn-1 and sn-3 positions, can be used to provide cocoa butter equivalents and components thereof.

Since the process of the invention involves the use of an sn-1,3-specific lipase, in order to provide a composition having a high level of SOS it is necessary that the vegetable oil already has a high level of oleic acid residues at the sn-2 position. Accordingly, the vegetable oil should have an oleic acid content in the sn-2 position of at least 75% by weight of the total sn-2 fatty acid residues of the vegetable oil provided in the reaction environment. Preferably, the vegetable oil has an oleic acid content in the sn-2 position of at least 80%, more preferably at least 85%, even more preferably at least 90%, and most preferably at least 95% by weight of the total sn-2 fatty acid residues of the vegetable oil provided in the reaction environment.

The oleic acid content at the sn-2 position of a vegetable oil can be determined by a method which involves scission of the fatty acids at the sn-1 and sn-3 positions using a pancreatic lipase enzyme followed by isolation of the resulting sn-2 monoacylglycerols (MAG) using TLC or NPLC and finally fatty acid methyl ester analysis by gas chromatography. A suitable method is IUPAC Official Method 2.210: “Determination of fatty acids in the 2-position in the triglycerides of oils and fats”, seventh ed., Standard Methods for the Analysis of Oils, Fats and Derivatives, Blackwell, Oxford, 1992.

In one or more embodiments, the vegetable oil (ii) is selected from High Oleic Rapeseed/canola oil, Olive oil, High Oleic Soybean oil, High Oleic Sunflower oil, High Oleic Safflower oil, Shea oil, or Rapeseed oil, and/or fractions or combinations thereof. Preferably the vegetable oil (ii) is selected from High Oleic Sunflower oil, High Oleic Safflower oil, and/or combinations or fractions thereof. These oils are particularly suitable, because they have a high oleic acid content and relatively low cost.

The reaction environment further comprises a source of stearic acid and/or palmitic acid residues, which is an aliphatic alcohol ester of stearic acid, palmitic acid, or mixtures thereof, optionally mixed with stearic acid and/or palmitic acid. The use of aliphatic alcohol esters of stearic and palmitic acid is advantageous, because these esters have lower melting points than the corresponding fatty acids. This enables lower temperatures to be used, which improves enzyme stability, whilst avoiding unwanted crystallization during processing. Preferably, the aliphatic alcohol ester of stearic acid or palmitic acid is an alkyl ester of stearic acid or palmitic ester or a mixture thereof, more preferably a C₁-C₄ alkyl ester of stearic acid or palmitic acid or a mixture thereof. Even more preferably, the ester is selected from the group consisting of methyl stearate, ethyl stearate, methyl palmitate, ethyl palmitate and mixtures thereof. Most preferably, the ester is selected from the group consisting of methyl stearate, ethyl stearate and mixtures thereof.

Stearate esters are preferred, because SOS triglycerides in which S represents stearic acid residues cannot be obtained in high amounts from widely available vegetable oils such as palm oil. The aliphatic alcohol ester of stearic acid or palmitic acid may be used in mixture with stearic acid and/or palmitic acid as free fatty acids. However, preferably, these free fatty acids are not used.

The reaction environment also comprises water. The presence of water is necessary to provide sufficient enzyme activity for high levels of SOS triglycerides to be obtained. However, when the level of water is too high, it has been found that diacylglycerides and SSO triglycerides are formed in higher amounts in the triglyceride phase which is obtained in step (b) of the process. It has been found that, to provide a high level of SOS triglycerides and a low level of diacylglycerides and SSO triglycerides, the water activity in step a) should be in the range 0.1-0.6, preferably 0.2-0.4.

The process of the present invention utilises a particularly high ratio of aliphatic alcohol ester and optional stearic acid and/or palmitic acid (iii) to vegetable oil (ii). It has been found that a high substrate ratio of at least 4:1 results in greater incorporation of stearic acid and palmitic acid in the sn-1 and sn-3 positions of the triglyceride, and therefore production of more SOS triglycerides. Surprisingly, the high substrate ratio, when combined with the other reaction conditions of process of the invention, does not increase the production of SSO and SSS triglycerides to an unacceptably high level. Accordingly, the triglyceride compositions which are produced by the process of the invention may be used as cocoa butter equivalents, or components thereof, without the need for further fractionation steps to separate SOS triglycerides from SSO and SSS triglycerides. Preferably, the substrate ratio is at least 5:1, more preferably at least 6:1, and even more preferably at least 7:1. Even higher substrate ratios are also contemplated within the present invention, such as at least 8:1 or at least 9:1. At very high substrate ratios, the efficiency of the process can be reduced by the need to remove large amounts of fatty acid ester and free fatty acids in step (c). Accordingly, the substrate ratio is typically at most 15:1, or at most 12:1, or at most 10:1.

The substrate ratio which is used in a particular process can be varied depending on the nature of the vegetable oil (ii). For example, when the vegetable oil has an oleic acid content in the sn-2 position which is particularly high, a high level of SOS triglycerides can be achieved by transesterification even when a substrate ratio is used which is towards the lower end of the specified range. In such cases, the use of substrate ratios towards the lower end of the specified range may be advantageous for reasons of process efficiency. Accordingly, in one embodiment, the vegetable oil has an oleic acid content in the sn-2 position of at least 90% or at least 95% by weight of the total sn-2 fatty acid residues of the vegetable oil and the weight ratio of the aliphatic alcohol ester and optional stearic acid and/or palmitic acid (iii) to the vegetable oil (ii) is in the range 4:1 to 7:1. Conversely, if the vegetable oil has an oleic acid content in the sn-2 position which is lower, then a higher substrate ratio can be used to maximise the level of SOS triglycerides in the reaction product. Accordingly, in another embodiment, the vegetable oil has an oleic acid content in the sn-2 position in the range 75% to 90%, or in the range 75% to 85% by weight of the total sn-2 fatty acid residues of the vegetable oil and the weight ratio of the aliphatic alcohol ester and optional stearic acid and/or palmitic acid (iii) to the vegetable oil (ii) is at least 7:1. Thus, the substrate ratio can be varied, depending on the triglyceride content of the vegetable oil which is used and the product composition which is desired.

In all embodiments, the temperature to which the reaction environment is heated is in the range 30-60° C. Preferably the temperature is in the range 35-45° C. The temperature of the reaction environment is chosen so as to be high enough to provide good enzyme activity and thus a high level of SOS triglycerides, whilst also being low enough to minimise the production of undesirable by-products, such as SSO and SSS triglycerides. The use of low reaction temperatures promotes enzyme stability, and is made possible by the use of aliphatic alcohol esters, which generally have low melting points.

The process of the invention can be carried out as a batch process, a fed-batch process, or a continuous process.

In a batch process, components (i)-(iv) are mixed in one reactor for a certain reaction time until desired yield of product is produced, and the enzymes are filtered off.

In a fed-batch process, the substrate is added to the batch reactor not all at once, but little at a time.

In a continuous process, the enzymes are fixed in a packed bed reactor, substrate is pumped through the reactor, and a product stream is drawn off from the reactor. In a continuous process, the flow rate of the feed (i.e. the sum of weight of the vegetable oil (ii) and the aliphatic alcohol ester of stearic acid, palmitic acid, or mixtures thereof, optionally mixed with stearic acid and/or palmitic acid (iii)) through the reactor is preferably in the range 0.5 to 14, more preferably 2 to 10, and most preferably 4 to 8 g feed/g enzyme/h. Continuous processes have been found to be advantageous for simpler recycling of fatty acid esters and fatty acids and reduced migration of acyl groups within the triglycerides. In advantageous embodiments of the invention, fatty acid esters and free fatty acids which remain in the reaction mixture after the transesterification are optionally processed and recirculated to the reaction environment.

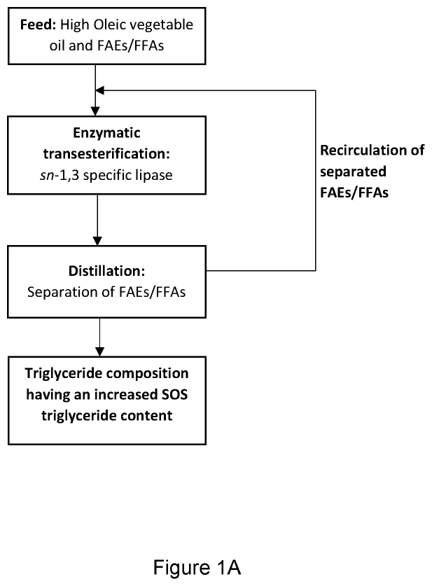

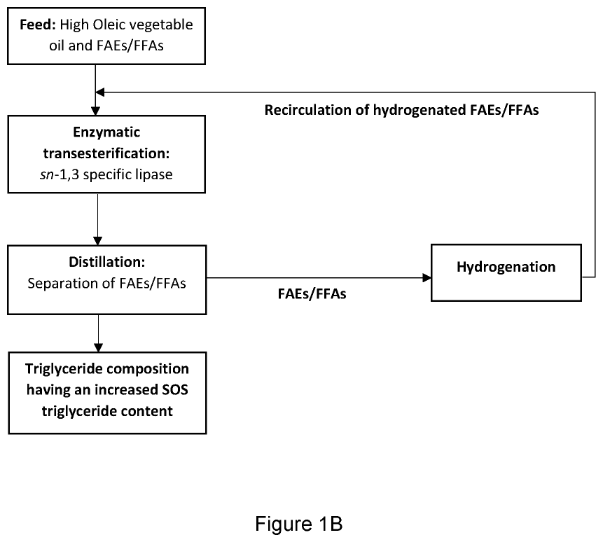

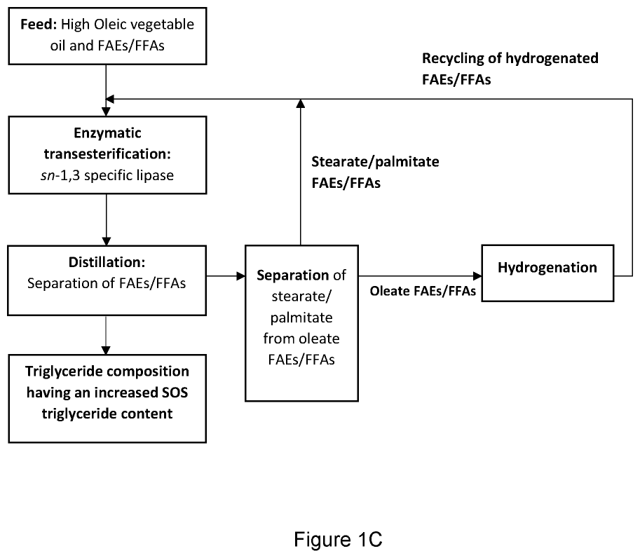

In one embodiment, the process further comprises a step (d1) comprising recirculating fatty acid esters and, where present, free fatty acids separated in step (c) to the reaction environment. In this way, a more efficient reaction can be achieved and the substrate ratio can be increased without the cost of providing additional reactants. Such a process is depicted schematically in FIG. 1A.

In an alternative embodiment, in which the aliphatic alcohol ester (iii) comprises an aliphatic alcohol ester of stearic acid optionally mixed with stearic acid, the process further comprises a step (d2) comprising hydrogenating fatty acid esters and, where present, free fatty acids separated in step (c) and recirculating the hydrogenated fatty acid esters and free fatty acids to the reaction environment. In this embodiment, free oleic acid and oleic acid esters, which are among the components separated in step (c) can be converted to stearic acid and esters thereof to further supplement the aliphatic alcohol esters of stearic acid and optional stearic acid in the reaction environment. This embodiment is depicted schematically in FIG. 1B.

In a further alternative embodiment, the process further comprises a step (d3) comprising separating fatty acid esters and, where present, free fatty acids separated in step (c) into a first fraction comprising stearate and/or palmitate ester, and optionally stearic acid and/or palmitic acid, and a second fraction comprising oleate ester and optionally oleic acid, and recirculating the first fraction to the reaction environment. Optionally, in this embodiment, the second fraction may be hydrogenated to provide stearic acid ester and optionally stearic acid and recirculated to the reaction environment. This embodiment is depicted schematically in FIG. 1C.

In an embodiment, the separated fatty acid esters and free fatty acids, i.e. the fatty acid esters and optionally free fatty acids separated in step (c), or the hydrogenated fatty acid esters and free fatty acids provided in step (d2), or the first fraction provided in step (d3), or the second fraction provided in step (d3) (either before or after hydrogenation), are bleached prior to being recirculated to the reaction environment. The bleaching removes impurities and secures a better enzymatic lifetime and hence a more efficient transesterification.

As discussed above, one advantage of the process of the invention is that the triglyceride composition which is produced has a sufficiently high level of SOS triglycerides and a sufficiently low level of SSO and SSS triglycerides to be used as a cocoa butter equivalent, or a component thereof, without the need for a further fractionation step. Accordingly, in a preferred embodiment, the triglyceride composition obtained in step (c) is not subjected to a fractionation step to provide a fractionated triglyceride composition having a further increased content of SOS triglycerides.

The process may also further comprise using the triglyceride composition obtained in step (c) as a cocoa butter equivalent or a component thereof in the manufacture of a chocolate or chocolate-like product. In an especially preferred embodiment, the triglyceride composition obtained in step (c) is not subjected to a fractionation step to provide a fractionated triglyceride composition having a further increased content of SOS triglycerides, and the triglyceride composition is used as a cocoa butter equivalent or a component thereof in the manufacture of a chocolate or chocolate-like product.

The invention also provides a triglyceride composition obtainable by the process of the invention. The triglyceride composition can be used as a component of a cocoa butter equivalent, typically in an amount of 10-70% by weight. Accordingly, the invention also provides a cocoa butter equivalent which comprises 10-70% by weight of the triglyceride composition. The invention also provides chocolate or chocolate-like products which comprise the triglyceride composition.

### EXAMPLES

In the embodiments where one may like to determine the individual positional isomers, e.g. the amounts of SOS, SSO, and SSS in the triglyceride phase, a Non-Aqueous Reversed-Phase HPLC method was used (Causevic, A. et al. “Non-aqueous reversed phase liquid chromatography with charged aerosol detection for quantitative lipid analysis with improved accuracy”, Journal of Chromatography A, Vol. 1652, 2021, pages 1-11,). The method was also used to separate and quantify different free fatty acids, fatty acid esters, monoacylglycerides, diacylglycerides and triacylglycerides.

### Example 1

Enzymatic transesterification was performed in a continuous process setup to produce SOS from a feed containing High Oleic Safflower oil (HOSFO) and Methyl Stearate (Me-St). Columns packed with 15 g of immobilized Lipase DF “Amano” IM from Rhizopus oryzae (Amano Enzyme) were used, together with reaction conditions described in the table below. The flow rate was controlled by a gear pump, pumping the oil from the tanks through the enzyme columns. The temperature was controlled and set by a heater, heating up the water bath where the substrate tanks, connections and enzymatic columns were placed.

| | Substrate | | | | | | |
|---|---|---|---|---|---|---|---|
| | ratio | | | Flow rate | SOS % in | SSO % in | |
| Experiment | (Me-St/ | Water | Temperature | (g oil/ | triglyceride | triglyceride | Ratio |
| No. | HOSFO) | activity | (° C.) | g enzyme/h) | phase | phase | SOS/SSO |
| 1 | 9.0 | 0.24 | 50.0 | 6.9 | 81.2 | 0.6 | 140 |
| 2 | 5.0 | 0.24 | 45.0 | 9.1 | 72.7 | 0.4 | 184 |
| 3 | 7.0 | 0.24 | 45.0 | 9.1 | 78.5 | 0.5 | 138 |
| 4 | 9.0 | 0.24 | 45.0 | 9.0 | 81.5 | 0.7 | 116 |
| 5 | 5.0 | 0.24 | 50.0 | 8.9 | 73.8 | 0.4 | 184 |
| 6 | 2.0 | 0.24 | 50.0 | 7.4 | 41.4 | 0.3 | 125 |
| 7 | 3.0 | 0.24 | 50.0 | 9.1 | 46.1 | 0.2 | 255 |
| 8 | 4.0 | 0.7 | 70.0 | 9.2 | 61.8 | 2.2 | 28 |

S = C18:0 and C16:0, O = C18:1

It can be seen from these results that using a high substrate ratio, together with the further process conditions of the invention, a high quality product can be produced, containing a high SOS content and a high ratio of SOS/SSO. In contrast, when the substrate ratio is low, as in Experiments 6 and 7, an SOS content above 65% is not achieved. Moreover, in Experiments 1-5, the SSO content of the triglyceride phase remains sufficiently low, such that the SOS/SSO ratio is acceptable. In contrast, when the temperature and water activity are high, as in Experiment 8, SSO triglycerides are produced in a higher amount, resulting in a low SOS/SSO ratio.

1. A process for increasing the SOS triglyceride content of a vegetable oil, wherein S represents stearic acid (C18:0) and palmitic acid (C16:0) residues and O represents oleic acid (C18:1) residues, said process comprising:
a) providing a reaction environment comprising:
i) an sn-1,3-specific lipase immobilised on a support,
ii) a vegetable oil, wherein the vegetable oil comprises at least 45% oleic acid fatty acid residues, based on total C6-C24 fatty acid residues, and wherein the vegetable oil has an oleic acid content in the sn-2 position of at least 75% by weight of the total sn-2 fatty acid residues of the vegetable oil,
iii) an aliphatic alcohol ester of stearic acid, palmitic acid, or mixtures thereof, optionally mixed with stearic acid and/or palmitic acid, and
iv) water;
wherein the weight ratio of the aliphatic alcohol ester and optional stearic acid and/or palmitic acid (iii) to the vegetable oil (ii) is at least 4:1; and
wherein the water activity of the reaction environment is in the range 0.1 to 0.6;
b) heating the reaction environment to a temperature of 30-60° C. to perform transesterification, thus obtaining a mixture comprising a triglyceride phase, fatty acid esters, and optionally free fatty acids; and
c) separating the fatty acid esters and free fatty acids from the mixture obtained in step (b) to obtain a triglyceride composition.

2. The process of claim 1, wherein the triglyceride composition obtained in step (c) is not subjected to a fractionation step to provide a fractionated triglyceride composition having a further increased content of SOS triglycerides.

3. The process of claim 1, wherein the triglyceride phase of the mixture obtained in step (b) has an SOS triglyceride content of at least 65% by weight of the triglyceride phase.

4. The process of claim 1, wherein the triglyceride phase of the mixture obtained in step (b) has a weight ratio of SOS triglycerides to SSO triglycerides of at least 80:1.

5. The process of claim 1, wherein the triglyceride phase of the mixture obtained in step (b) has an SSO content of ≤1.2% by weight of the triglyceride phase.

6. The process of claim 1, wherein the water activity of the reaction environment is in the range 0.2 to 0.4.

7. The process of claim 1, wherein the vegetable oil comprises at least 50% oleic acid (C18:1) fatty acid residues based on total C6-C24 fatty acid residues.

8. The process of claim 1, wherein the vegetable oil has an oleic acid content in the sn-2 position of at least 80% by weight of the total sn-2 fatty acid residues of the vegetable oil.

9. The process of claim 1, wherein the vegetable oil is selected from High Oleic Rapeseed/canola oil, Olive oil, High Oleic Soybean oil, High Oleic Sunflower oil, High Oleic Safflower oil, Shea oil, or Rapeseed oil and/or fractions or combinations thereof.

10. The process of claim 1, wherein the vegetable oil is selected from High Oleic Sunflower oil, High Oleic Safflower oil and/or fractions or combinations thereof.

11. The process of claim 1, wherein the sn-1,3-specific lipase is a microbial lipase, such as a bacterial lipase or a fungal lipase.

12. The process of claim 11, wherein the sn-1,3-specific lipase is derived from a fungal species selected from Rhizopus oryzae, Thermomyces lanuginosus, and Rhizomucor miehei.

13. The process of claim 1, wherein the reaction environment is heated to a temperature of 35-45° C.

14. The process of claim 1, wherein the weight ratio of the aliphatic alcohol ester and optional stearic acid and/or palmitic acid (iii) to the vegetable oil (ii) is at least 5:1, such as at least 6:1 or at least 7:1.

15. The process of claim 1, wherein the vegetable oil has an oleic acid content in the sn-2 position of at least 90%, preferably at least 95% by weight of the total sn-2 fatty acid residues of the vegetable oil, and the weight ratio of the aliphatic alcohol ester and optional stearic acid and/or palmitic acid (iii) to the vegetable oil (ii) is in the range 4:1 to 7:1.

16. The process of claim 1, wherein the aliphatic alcohol ester of stearic acid or palmitic acid is an alkyl ester of stearic acid or palmitic acid or a mixture thereof.

17. The process of claim 1, wherein the transesterification is performed as a batch process, as a fed-batch process, or as a continuous process.

18. The process of claim 1, further comprising:
d1) recirculating fatty acid esters and, where present, free fatty acids separated in step (c) to the reaction environment.

19. The process of claim 1, wherein the aliphatic alcohol ester (iii) comprises an aliphatic alcohol ester of stearic acid, optionally mixed with stearic acid, and wherein the process further comprises:
d2) hydrogenating fatty acid esters and, where present, free fatty acids separated in step (c) and recirculating the hydrogenated fatty acid esters and free fatty acids to the reaction environment.

20. The process of claim 1, further comprising:
d3) separating fatty acid esters and, where present, free fatty acids separated in step (c) into:
a first fraction comprising stearate and/or palmitate ester, and optionally stearic acid and/or palmitic acid; and
a second fraction comprising oleate ester and optionally oleic acid; and recirculating the first fraction to the reaction environment.

21. The process of claim 20, wherein the aliphatic alcohol ester (iii) comprises an aliphatic alcohol ester of stearic acid, optionally mixed with stearic acid, and wherein the process further comprises:
e3) hydrogenating the second fraction to provide stearate ester and optionally stearic acid and recirculating the hydrogenated second fraction to the reaction environment.

22. The process of claim 1, wherein any of the following are bleached before being recirculated to the reaction environment:
the fatty acid esters and optionally free fatty acids separated in step (c);
the hydrogenated fatty acid esters and free fatty acids provided in step (d2);
the first fraction provided in step (d3); or
the second fraction provided in step (d3), either before or after hydrogenation.

23. The process of claim 1, further comprising using the triglyceride composition obtained in step (c) as a cocoa butter equivalent or a component thereof in the manufacture of a chocolate or chocolate-like product.

24. A triglyceride composition obtainable by the process of claim 1.

25. A cocoa butter equivalent which comprises 10-70% by weight of the triglyceride composition of claim 24.

26. A chocolate or chocolate-like product comprising the triglyceride composition of claim 24.
